(19) United States
(12) Patent Application Publication
(10) Pub. No.: US 2012/0214778 A1
(43) Pub. Date: Aug. 23, 2012

(54) FULVESTRANT IN A DOSAGE OF 500mg FOR THE TREATMENT OF ADVANCED BREAST CANCER

(76) Inventors: Isaiah William Dimery, Wilmington, DE (US); Alan Webster, Macclesfield (GB)

(21) Appl. No.: 13/387,584

(22) PCT Filed: Jul. 26, 2010
(86) PCT No.: PCT/GB2010/051228

§ 371 Date: May 7, 2012

(30) Foreign Application Priority Data
Jul. 27, 2009 (GB) 0912999.0

(51) Int. Cl. A61K 31/565; A61P 35/00

(52) U.S. Cl. 514/182

(57) ABSTRACT

The present disclosure is directed to fulvestrant at a dosage of 500 mg and its use in the treatment of a postmenopausal woman with advanced breast cancer.

The present invention relates to fulvestrant at a dosage of 500 mg for use in the treatment of a postmenopausal woman with advanced breast cancer who has progressed or recurred on endocrine therapy.

Breast cancer is one of the most common malignancies in women, comprising 18% of female cancers worldwide (Mcpherson et al 2000), and the most common cause of cancer deaths. The incidence varies among populations with about half of all cases occurring in North America and Western Europe. It has long been acknowledged that many breast cancers are hormone dependent and that hormonal manipulation can affect the progress of the disease (Beatson 1896). The most important factor determining response to hormonal manipulation is the presence of the oestrogen receptor (ER) in the target tissue (Fisher et al 2001).

The antioestrogen (AO) tamoxifen has been the most widely used endocrine therapy for breast cancer in both premenopausal and postmenopausal women. However, despite its demonstrated efficacy, de novo or acquired resistance may occur during treatment. In some patients, the disease progresses during therapy because tumour growth may be stimulated by tamoxifen, due to its partial agonist activity on the ER (Wiebe et al 1993).

The search for a pure AO, devoid of the agonist activity of tamoxifen, resulted in the discovery and clinical development of ICI 182,780 (also known as fulvestrant or FASLODEX™). Fulvestrant is an ER antagonist without known agonistic properties that down-regulates cellular levels of the ER in a dose-dependent manner (Howell et al 2000, Robertson et al 2001, Wakeling et al 1991). Fulvestrant is well tolerated and has demonstrated efficacy in women whose breast cancer had progressed following endocrine therapy (Howell et al 2002, Osborne et al 2002, Chia et al 2008).

Women diagnosed with early breast cancer are generally treated with tamoxifen or an aromatase inhibitor if endocrine therapy is appropriate. However if the cancer recurs or progresses there is a need for alternative therapies. Fulvestrant (FASLODEX™) is presently approved at a dose of 250 mg as an alternative endocrine therapy. The present invention is based on the discovery that increasing the dose of fulvestrant to 500 mg is more advantageous for patients than the 250 mg dose.

One feature of the invention provides fulvestrant at a dosage of 500 mg for use in the treatment of a postmenopausal woman with advanced breast cancer who has progressed or recurred on endocrine therapy. Preferably the fulvestrant is administered monthly. Preferably an additional dose of 500 mg is administered during the first month of treatment. Preferably the additional dose is administered at about day 14. Preferably the woman is oestrogen receptor positive or progesterone receptor positive; more preferably oestrogen receptor positive. Preferably the progression or recurrence on endocrine therapy comprised therapy with tamoxifen or an aromatase inhibitor. Preferably the aromatase inhibitor is selected from anastrozole, letrozole or exemestane; more preferably anastrozole or letrozole. Preferably the use use of fulvestrant at 500 mg dosage provides an increase the time to progression compared with fulvestrant at a dosage of 250 mg; in particular the doses are preferably administered monthly with an additional dose at 500 mg in the first month. Tamoxifen, anastrozole, letrozole and exemestane are all commercially available drugs with regulatory approval for administration to women with breast cancer.

Another feature of the invention provides the use fulvestrant at a dosage of 500 mg for preparation of a medicament for treatment of a postmenopausal woman with advanced breast cancer who has progressed or recurred on endocrine therapy. This feature may be combined with any of the preferred features described herein.

Another feature of the invention provides the treatment of a postmenopausal woman with advanced breast cancer who has progressed or recurred on endocrine therapy with fulvestrant at a dosage of 500 mg. This feature may be combined with any of the preferred features described herein.

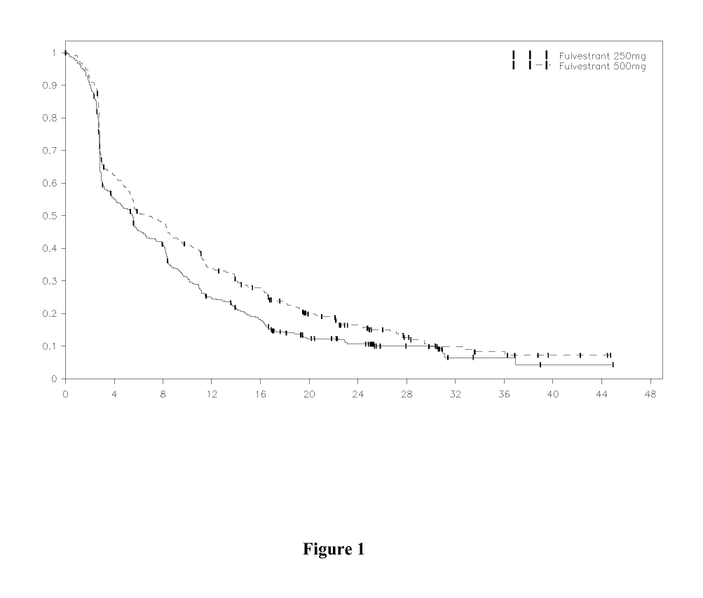

The invention is exemplified by the following non-limiting Example, in which FIG. 1 shows a Kaplan-Meier plot of time to progression comparing fulvestrant at 250 mg with 500 mg. The x-axis shows the time in months and y-axis shows proportion of patients progression free. Tick marks indicate censored observations.

| Abbreviation or | |
|---|---|
| special term | Explanation |
| AE | Adverse event |
| AI | Aromatase inhibitor |
| ALT | Alanine aminotransferase |
| AO | Antioestrogen |
| AST | Aspartate aminotransferase |
| BOR | Best objective/overall response |
| CBR | Clinical benefit rate |
| CI | Confidence interval |
| CR | Complete response |
| CRA | Clinical research associate |
| CRF | Case report form |
| CSP | Clinical Study Protocol |
| CSR | Clinical Study Report |
| CT | Computed tomography |
| CTCAE | Common terminology criteria for adverse events |
| DAE | Premature discontinuation of treatment with |
| | investigational product due to an adverse event |
| | (adverse events). |
| DCO | Data cut-off |
| DoCB | Duration of clinical benefit |
| DoR | Duration of response |
| ECG | Electrocardiogram |
| EDoCB | Expected duration of clinical benefit |
| EDoR | Expected duration of response |
| Endpoint | A status of the patient that constitutes the |
| | ‘endpoint’ of a patient's participation in a |
| | clinical study and that is used as the final outcome. |
| ER | Oestrogen receptor |
| EU | European Union |
| FACT-B | Functional Assessment of Cancer Therapy - |
| | breast cancer |
| FSH | Follicle stimulating hormone |
| GCP | Good clinical practice |
| HER | Human epidermal growth factor receptor |
| HRQoL | Health-related quality of life |
| ICH | International Conference on Harmonisation |
| IDMC | Independent Data Monitoring Committee |
| IEC | Independent Ethics Committee |
| im | Intramuscular |
| INK | International normalised ratio |
| IRB | Institutional Review Board |
| International | An Investigator assigned the responsibility for the |
| Co-ordinating | co-ordination of investigators across all Study |
| investigator | Sites participating in a multinational, multicentre study. |
| LD | Longest diameter |
| LHRH | Luteinising hormone releasing hormone |
| MedDRA | Medical dictionary for regulatory activities |
| MRI | Magnetic resonance imaging |
| NCCN | National Comprehensive Cancer Network |
| OAE | Other significant adverse event (ie, significant AEs, |
| | other than SAEs and DAEs, which are of particular |
| | clinical importance in this development program). |
| OR | Objective response |
| ORR | Objective response rate |
| OS | Overall survival |
| Outcome | A variable (usually a derived variable) specifically |
| variable | defined to be used in the analysis of a study objective. |
| Patient | Only one variable is used to identify each patient |
| identifier | within the reporting database. This identifier is |
| | a concatenation of the Study Number, and the enrolment |
| | Code (eg, D1234C00001/E0010001). Within an |
| | individual study report, the enrolment code alone |
| | (eg, E0010001) may be used to reference individual |
| | patients in-text within the CSR, including tables and |
| | listings. With respect to individual Patient Narratives, |
| | and the higher level documents, the full unique patient |
| | identifier should be used. |
| PD | Progressive disease |
| PgR | Progesterone receptor |
| PPS | Per Protocol Set |
| PR | Partial response |
| Principal | A person responsible for the conduct of a clinical |
| investigator | study at an investigational study site. Every |
| | investigational study site has a principal investigator. |
| PRO | Patient reported outcomes |
| PT | Preferred term |
| RECIST | Response evaluation criteria in solid tumours |
| SAE | Serious adverse event |
| SAP | Statistical Analysis Plan |
| SD | Stable disease |
| sd | Standard deviation |
| SE | Standard error |
| SOC | System organ class |
| TOI | Trial outcome index |
| TTP | Time to progression. The definition of TTP used in |
| | this clinical study is also commonly termed |
| | progression free survival (PFS). |
| TTR | Time to response |
| ULRR | Upper limit reference range |
| US | United States of America |
| Variable | A characteristic or a property of a patient that may |
| | vary eg from time to time or between patients. |
| WHO | World Health Organisation |

### EXAMPLE 1

### A Randomised, Double-Blind, Parallel-Group, Multicentre, Phase III Study Comparing the Efficacy and Tolerability of Fulvestrant (FASLODEX™) 500 mg with Fulvestrant (FASLODEX™) 250 mg in Postmenopausal Women with Oestrogen Receptor Positive Advanced Breast Cancer Progressing or Relapsing after Previous Endocrine Therapy

This study assessed the relationship between fulvestrant dose and efficacy. It compared the current approved dose and dosing schedule of fulvestrant (250 mg every 28 days) with a higher dose regimen (500 mg every 28 days plus an additional 500 mg on Day 14 of the first month only). The study is also referred to as CONFIRM.

Study Centres

One-hundred and twenty-eight centres in 17 countries (Belgium, Brazil, Chile, Colombia, Czech Republic, Hungary, India, Italy, Malta, Mexico, Poland, Russia, Slovakia, Spain, USA, Ukraine and Venezuela). The US, Mexico, Italy, Brazil, Spain, Chile, Colombia and Venezuela also participated in health-related quality of life (HRQoL) assessments during the study.

Objectives

The primary objective of the study was to compare the efficacy of fulvestrant 500 mg treatment with fulvestrant 250 mg treatment in terms of time to progression (TTP). The secondary objectives of the study were:
To compare the objective response rate (ORR) of patients treated with fulvestrant 500 mg with the objective response rate of patients treated with fulvestrant 250 mg.
To compare clinical benefit rate (CBR) of patients treated with fulvestrant 500 mg with the clinical benefit rate of patients treated with fulvestrant 250 mg.
To compare duration of response (DoR) of patients treated with fulvestrant 500 mg with the duration of response of patients treated with fulvestrant 250 mg.
To compare the duration of clinical benefit (DoCB) of patients treated with fulvestrant 500 mg with the duration of clinical benefit of patients treated with fulvestrant 250 mg.
To compare the overall survival (OS) of patients treated with fulvestrant 500 mg with the overall survival of patients treated with fulvestrant 250 mg.
To assess the tolerability of fulvestrant 500 mg treatment compared with fulvestrant 250 mg treatment.
To assess the health-related quality of life (HRQoL) of patients treated with fulvestrant 500 mg as compared to fulvestrant 250 mg in a subgroup of patients.

Study Design

This was a randomised, double-blind, parallel-group, multicentre, phase III study to compare 2 dose levels of fulvestrant in postmenopausal women with oestrogen receptor positive (ER+ve) advanced breast cancer who had either relapsed whilst on adjuvant endocrine therapy, or progressed whilst on first endocrine therapy for advanced disease.

Target Patient Population and Sample Size

A total of 720 postmenopausal women with histological/cytological confirmation of ER+ve breast cancer who had relapsed or progressed on previous endocrine therapy were planned to be recruited; a total of 736 were actually randomised.

The sample size calculation was based on the primary variable, TTP, and assumed exponential progression times. The sample size was driven by the number of required events. In order to detect a hazard ratio of ≦0.8 (or ≧1.25) for fulvestrant 500 mg compared to fulvestrant 250 mg, at a 2-sided significance level of 5%, with 80% power, approximately 632 events were required to have occurred in the study (ie, approximately 632 patients to have progressed or died).

Investigational Product and Comparator: Dosage, Mode of Administration and Batch N

umbers Fulvestrant 500 mg was given as two 5 ml intramuscular (im) injections, one in each buttock, on days 0, 14, 28 and every 28 (±3) days thereafter.

Fulvestrant 250 mg was given as two 5 ml im injections (1 fulvestrant injection plus 1 placebo injection), one in each buttock, on days 0, 14 (2 placebo injections only), 28 and every 28 (±3) days thereafter.

Duration of Treatment

Treatment was to continue until disease progression occurred, unless any of the criteria for treatment discontinuation were met first.

Criteria for Evaluation—Efficacy and Pharmacokinetics (Main Variables) Efficacy

The primary outcome variable TTP; secondary variables were ORR, CBR, DoR, DoCB and OS.

Patient Reported Outcomes

The primary patient reported outcome for HRQoL was the Trial Outcome Index (TOI) derived from the Functional Assessment of Cancer Therapy—Breast cancer (FACT-B) questionnaire.

Criteria for Evaluation—Safety (Main Variables)

Outcome variables for safety were frequency and severity of adverse events (AEs), including pre-specified AEs of interest.

Statistical Methods

For the primary endpoint TTP, the primary analysis was an unadjusted log-rank test and the secondary analysis was a Cox proportional hazard model, adjusted for treatment and other predefined covariates.

For OS, the unadjusted log-rank test was performed. For ORR and CBR, a logistic regression model with treatment factor only was fitted. DoR and DoCB were analysed in those patients who had an OR and CB, respectively. For HRQoL endpoints, a longitudinal model with treatment and other covariates was used.

The hypotheses for TTP, ORR, CBR, DoR, DoCB, OS, FACT-B score and TOI score were:

H₀: fulvestrant 500 mg is not different from fulvestrant 250 mg, vs.

H₁: fulvestrant 500 mg is different from fulvestrant 250 mg

For efficacy and HRQoL endpoints, summaries and analyses were carried out according to the randomised treatment i.e., using the Full Analysis Set. For safety endpoints, summaries and analyses were carried out according to the treatment actually received, i.e., using the safety analysis set. The primary endpoint was also analysed in the per protocol set (PPS). Patient population

A total of 720 patients were planned to be recruited; 736 were actually randomised. Diagram S1 shows the number of patients randomised to each of the 2 treatment groups and the number in each of the populations analysed. In addition, HRQoL was analysed in 145 of the patients in the Full Analysis Set (72 patients in the fulvestrant 500 mg group and 73 patients in the fulvestrant 250 mg group). The patient population was consistent with the one intended to be recruited. In the fulvestrant 500 mg group, 41 patients were ongoing study treatment at data cut off (DCO) compared with 31 patients in the fulvestrant 250 mg group. 1.1 Selection of Study Population

Before entering the study, patients were assessed to ensure that they met the eligibility criteria. Investigators had to keep a record of patients who were considered for enrollment but were never randomised (patient screening log). This information is necessary to establish that the patient population was selected without bias. The patient screening log had to be filed in the Investigator study file at each centre.

1.1.1 Inclusion Criteria

For inclusion in the study patients had to fulfil all of the following criteria:
1. Provision of written informed consent
2. Histological/cytological confirmation of breast cancer
3. Documented ER+ve status of primary or metastatic tumour tissue, according to the local laboratory parameters
4. Requiring endocrine therapy:
   Relapsing during, or within 12 months of completion of, adjuvant endocrine therapy (tamoxifen, toremifene or AIs such as anastrozole, letrozole and exemestane), or
   Progressing on an endocrine therapy (tamoxifen, toremifene or AIs such as anastrozole, letrozole and exemestane) provided that this endocrine treatment was started at least 12 months after the completion of adjuvant endocrine treatment, or
   Progressing on an endocrine therapy (tamoxifen, toremifene or AIs such as anastrozole, letrozole and exemestane) given as first treatment for patients with de novo advanced¹ breast cancer Advanced breast cancer: Metastatic disease or locally advanced disease which is not amenable to treatment with curative intent. administered more than 4 months prior to randomisation, menses must not have restarted, and FSH and oestradiol levels must also have been in the postmenopausal range (utilising ranges from the local laboratory facility).
5. Fulfilling one of the following criteria:
   Patients with measurable disease as per RECIST criteria. This is defined as at least one lesion that can be accurately measured in at least one dimension (longest diameter to be recorded) as ≧20 mm with conventional techniques or as ≧10 mm with spiral CT scan.
   Patients with bone lesions, lytic or mixed (lytic and sclerotic), in the absence of measurable disease as defined by RECIST.
6. Postmenopausal woman, defined as a woman fulfilling any 1 of the following criteria:
   Age ≧60 years.
   Age ≧45 years with amenorrhoea ≧12 months with an intact uterus.
   Having undergone a bilateral oophorectomy
   Follicle stimulating hormone (FSH) and oestradiol levels in postmenopausal range (utilising ranges from the local laboratory facility).
   In patients who had previously been treated with a luteinising hormone releasing hormone (LHRH) analogue, the last depot must have been
7. WHO performance status 0, 1 or 2.

Rationale for Inclusion Criteria
1. This criterion was set as part of the ethical conduct of the study, which complies with GCP.
2. This criterion was set to objectively confirm breast cancer.
3. This criterion was set to select a patient population expected to respond to fulvestrant based on its mechanism of action.
4. This criterion was set to clarify the history of hormonal therapy for breast cancer in this study.
5. This criterion was set to enable the conduct of efficacy assessments according to modified RECIST.
6. This criterion was set because the effect of fulvestrant on pre-menopausal breast cancer patients had not been fully assessed.
7. This criterion was set to conduct efficacy assessments properly and to ensure the safety of patients.

1.1.2 Exclusion Criteria

Any of the following was regarded as a criterion for exclusion from the study:
1. Presence of life-threatening metastatic visceral disease, defined as extensive hepatic involvement, or any degree of brain or leptomeningeal involvement (past or present), or symptomatic pulmonary lymphangitic spread. Patients with discrete pulmonary parenchymal metastases were eligible, provided their respiratory function was not compromised as a result of disease.
2. More than one regimen of chemotherapy for advanced disease.² Patients previously treated with one regimen of chemotherapy for advanced disease were allowed as long as their last treatment was an AO or an AI.
3. More than one regimen of endocrine therapy for advanced disease.³ Oophorectomy, ovarian ablation, or LHRH analogue therapy did not count as endocrine treatments in this context and also did not render the patient ineligible for this study.
4. Extensive radiation therapy within the last 4 weeks (greater than or equal to 30% marrow or whole pelvis or spine) or cytotoxic treatment within the past 4 weeks prior to screening laboratory assessment, or strontium-90 (or other radiopharmaceuticals) within the past 3 months.
5. Treatment with a non-approved or experimental drug within 4 weeks before randomisation.
6. Current or prior malignancy within previous 3 years (other than breast cancer or adequately treated basal cell or squamous cell carcinoma of the skin or in-situ carcinoma of the cervix).
7. Any of the following laboratory values:
   Platelets <100×10⁹/L
   Total bilirubin >1.5×upper limit reference range (ULRR)
   ALT or AST >2.5×ULRR if no demonstrable liver metastases or >5×ULRR in presence of liver metastases.
8. History of:
   Bleeding diathesis (i.e., disseminated intravascular coagulation, clotting factor deficiency), or
   Long-term anticoagulant therapy (other than antiplatelet therapy and low dose warfarin (see Section 3.7 of the CSP [Appendix 12.1.1 of this report]).
9. History of hypersensitivity to active or inactive excipients of fulvestrant and/or castor oil.
10. Any severe concomitant condition which made it undesirable for the patient to participate in the trial or which would jeopardize compliance with the CSP, e.g., uncontrolled cardiac disease or uncontrolled diabetes mellitus.

Rationale for Exclusion Criteria

The exclusion criteria for concurrent diseases, concomitant drugs and patients' conditions were set because they were considered to affect the safety of patients or the efficacy assessment of fulvestrant in hormone receptor positive, postmenopausal advanced or recurrent breast cancer.

1.1.3 Restrictions

The following restrictions were applied to patients in this trial:
1. Patients who were blood donors were not to donate blood during the study and for 12 weeks following their last dose of randomised treatment.
2. Patients who had confirmed disease progression must have been discontinued from their randomised treatment.
3. Concomitant treatments listed in Section 3.7 of the CSP.

Rationale for Restrictions
1. This restriction was included to ensure that anaemia was not induced by blood donation following the additional blood sampling requirement of the study.
2. This restriction was included to protect patients who were not receiving or who ceased to receive clinical benefit from their study treatment and is in line with current clinical practice.
3. This restriction was included because the concomitant treatments listed in Section 3.7 of the CSP were considered to effect the safety of patients or the efficacy assessment of the study drugs.

1.1.4 Discontinuation of patients from treatment or assessment

Patients could be discontinued from study treatment and assessments at any time at the discretion of the investigators. Patients were also free to discontinue their participation in the study at any time, without prejudice to further treatment. Specific reasons for discontinuing a patient from this study, and the procedures to be followed when a patient discontinued or was incorrectly enrolled, are listed in Section 3.3.5 of the CSP. For patients who discontinued, it was noted whether they were assessed after study medication was stopped, and whether they were asked about the reason(s) for their discontinuation and about the presence of any adverse events (AEs). If possible, they were seen and assessed by an investigator. AEs were followed up for 56 days after the last injection.

Summary of Demographics and Baseline Characteristics

A total of 96.1% of patients randomised into the study were Caucasian. The mean age of patients was 60.9 years and the mean weight of patients was approximately 70 kg. Tumour characteristics were well balanced across the 2 treatment groups. Most patients (507 [68.9%]) were ER+ve and PgR+ve at primary diagnosis and almost all patients (721 [98%]) had metastatic disease at baseline. In this study, 42.5% of patients had relapsed or progressed on AI therapy and 57.5% had relapsed or progressed on AOs. Most patients had relapsed or progressed either during previous adjuvant endocrine cancer therapy (344 patients [46.7%]) or during endocrine therapy given as a first treatment for de novo advanced disease (255 patients [34.6%]). Approximately two thirds of patients had shown a response⁴ to their last endocrine therapy. Defined as patients who experienced recurrence after ≧2 years on adjuvant endocrine therapy and/or patients who received clinical benefit (CR, PR or SD ≧24 weeks) from first-line therapy for advanced disease.

Summary of Efficacy Results

A summary of efficacy data is presented in Table S1.

TABLE S1
Summary of efficacy results for the main outcome variables

| | |
|---|---|
| Variable | Result |

Primary outcome variable

| | |
|---|---|
| TTP^{a} | Hazard ratio = 0.80 (95% CI 0.68-0.94); p = 0.006 |
| | Median TTP: fulvestrant 500 mg = 6.5 months; fulvestrant |
| | 250 mg = 5.5 months |
| | % patients progression free at 12 months: fulvestrant |
| | 500 mg = 34%; fulvestrant 250 mg = 25% |

Secondary outcome variables

| | |
|---|---|
| ORR | Odds ratio = 0.94 (95% CI 0.57-1.55); p = 0.795 |
| | ORR: fulvestrant 500 mg = 13.8%; fulvestrant |
| | 250 mg = 14.6% |
| CBR | Odds ratio = 1.28 (95% CI 0.95-1.71); p = 0.100 |
| | CBR: fulvestrant 500 mg = 45.6%; fulvestrant |
| | 250 mg = 39.6% |
| DoR^{b} | Ratio of EDoR = 0.894 (95% CI 0.479-1.667); p = 0.724 |
| | Median DoR^{c}: fulvestrant 500 mg = 19.4 months; fulvestrant |
| | 250 mg = 16.4 months |
| DoCB | Ratio of EDoCB = 1.357 (95% CI 1.067-1.726); p = 0.013 |
| | Median DoCB: fulvestrant 500 mg = 16.6 months; fulvestrant |
| | 250 mg = 13.9 months |
| OS | Hazard ratio = 0.84 (95% CI 0.69-1.03); p = 0.091 |
| | Median OS: fulvestrant 500 mg = 25.1 months; fulvestrant |
| | 250 mg = 22.8 months |
| | % patients alive at 24 months: fulvestrant 500 mg = 53%; |
| | fulvestrant 250 mg = 49% |

^{a}TTP = progression-free survival. At data cut-off, 84% of patients had progressed or died in the absence of progression.
^{b}measured from randomisation to progression
^{c}from randomisation.

TTP:time to progression; ORR:objective response rate; CBR:clinical benefit rate; DoR:duration of response; DoCB:duration of clinical benefit; OS:overall survival; EDoR:expected duration of response; EDoCB:expected duration of clinical benefit. Fulvestrant 500 mg was associated with a significantly longer TTP compared with fulvestrant 250 mg (hazard ratio=0.80 [95% CI 0.68-0.94]; p=0.006) corresponding to a reduction in risk of progression of 20%. Subgroup analyses showed a consistent treatment effect across all 6 predefined baseline covariates, including patients treated previously with either an aromatase inhibitor (AI) or antioestrogen (AO).

The ORR for fulvestrant 500 mg and fulvestrant 250 mg were similar (13.8% and 14.6% respectively, odds ratio=0.94 [95% CI 0.57 to 1.55]; p=0.795) but there was a trend for an increased CBR in patients receiving fulvestrant 500 mg compared to those receiving fulvestrant 250 mg (45.6% vs. 39.6%, odds ratio=1.28 [95% CI 0.95 to 1.71]; p=0.100). There was no statistically significant difference between the 2 treatment groups in expected DoR (EDoR); however, there was a statistically significant improvement in expected DoCB (EDoCB) in patients randomised to receive fulvestrant 500 mg compared with patients randomised to receive fulvestrant 250 mg (9.83 months vs. 7.24 months, ratio of EDoCB=1.357 [95% CI 1.067 to 1.726]; p=0.013).

There was a trend for improved survival for patients treated with fulvestrant 500 mg compared with fulvestrant 250 mg (hazard ratio=0.84 [95% CI 0.69 to 1.03]; p=0.091); this corresponds to a 16% reduction in risk of death.

In the subgroup of patients where it was measured, on-treatment HRQoL for both fulvestrant 500 mg and fulvestrant 250 mg was good (mean TOI score of approximately 60 out of 92). Patients treated with fulvestrant 500 mg had a similar on-treatment HRQoL to patients treated with fulvestrant 250 mg and there were no statistically significant differences between the 2 treatment groups in terms of change in on treatment HRQoL as measured by both the TOI and FACT-B score, although there was a numerical advantage in TOI in favour of fulvestrant 500 mg.

Efficacy Results

Primary Variable: Time to Progression

The primary objective of this study was to compare TTP between patients treated with fulvestrant 500 mg and those treated with fulvestrant 250 mg. The primary analysis set was the Full Analysis Set. An analysis of TTP in the PPS was also performed as a secondary analysis. Table S2 shows the TTP data for patients in the fulvestrant 500 mg and fulvestrant 250 mg groups in the Full Analysis Set; FIG. 1 shows a Kaplan-Meier plot of these data.

At DCO 618/736 (84.0%) patients had progressed or died in the absence of progression (297 [82.0%] in the fulvestrant 500 mg group and 321 [85.8%] in the fulvestrant 250 mg group). The unadjusted log rank test indicates that the TTP for patients in the fulvestrant 500 mg group was significantly longer than for those in the fulvestrant 250 mg group (hazard ratio=0.80 [95% CI 0.68 to 0.94]; p=0.006). Median TTP was 6.5 months in the fulvestrant 500 mg group and 5.5 months in the fulvestrant 250 mg group. The Kaplan-Meier plot for TTP in the Full Analysis Set shows a separation between the 2 treatment groups from approximately 3 months, favouring the fulvestrant 500 mg group.

| Month | 0 | 4 | 8 | 12 | 16 | 20 | 24 | 28 | 32 | 36 | 40 | 44 | 48 |
|---|---|---|---|---|---|---|---|---|---|---|---|---|---|
| Fulvestrant | 362 | 216 | 163 | 113 | 90 | 54 | 37 | 19 | 12 | 7 | 3 | 2 | 0 |
| 500 mg at risk | | | | | | | | | | | | | |
| Fulvestrant | 374 | 199 | 144 | 85 | 60 | 35 | 25 | 12 | 4 | 3 | 1 | 1 | 0 |
| 250 mg at risk | | | | | | | | | | | | | |

TABLE S2
Summary of time to progression: Full Analysis Set

| | Fulvestrant 500 mg | Fulvestrant 250 mg |
|---|---|---|
| | N = 362 | N = 374 |
| Number progressed (%) | 297 (82.0) | 321 (85.8) |
| Median (months) | 6.5 | 5.5 |
| Time to progression | 2.8 | 2.7 |
| (months): 25% quartile | | |
| Time to progression | 16.6 | 11.9 |
| (months): 75% quartile | | |
| Percentage of patients | | |
| progression free at: | | |
| 6 months | 51% | 45% |
| 12 months | 34% | 25% |
| 18 months | 23% | 14% |
| 24 months | 16% | 11% |

| | |
|---|---|
| Hazard ratio (95% CI) | 0.80 (0.68-0.94) |
| p-value | 0.006 |

Time to progression is the time between randomisation and the earliest of progression or death from any cause.

A hazard ratio <1 indicates fulvestrant 500 mg is associated with a longer time to disease progression than fulvestrant 250 mg

A hazard ratio >1 indicates fulvestrant 500 mg is associated with a shorter time to disease progression than fulvestrant 250 mg

Data source: Tables 11.2.1.1, 11.2.1.2 and 11.2.1.5.

The primary analysis of TTP is supported by the Cox proportional hazards regression analysis, adjusted for treatment and 6 specified covariates (hazard ratio=0.78 [95% CI 0.67 to 0.92]; p=0.003).

Summary of Safety Results

Fulvestrant 500 mg was well tolerated and its safety profile was consistent with the known safety profile of fulvestrant 250 mg. The most commonly reported pre-specified AEs of interest were gastrointestinal disturbances and joint disorders (approximately 20% and 19% of patients, respectively, in each of the treatment groups). There were no differences between treatment groups in the incidence or type of AEs, serious AEs and AEs leading to discontinuation. There was no evidence for dose dependence for any AE. There were no clinically important changes in haematology, clinical chemistry, vital signs or physical findings.

Conclusions

This study demonstrates that fulvestrant 500 mg provides a clinically meaningful benefit over fulvestrant 250 mg, in terms of TTP, in the treatment of postmenopausal women with ER+ve advanced breast cancer who have progressed or recurred on endocrine therapy. Further analyses demonstrated that the TTP data obtained in the study are robust. The results show that fulvestrant 500 mg reduces the risk of disease progression by 20% compared with fulvestrant 250 mg. The risk in progression appears to be reduced in the fulvestrant 500 mg group compared to the 250 mg group by 3 observed factors:
a reduction in the proportion of patients with a best objective response of progressive disease (38.7% in the fulvestrant 500 mg group vs 44.7% in the fulvestrant 250 mg group)
an increase in the proportion of patients who achieved clinical benefit (45.6% vs 39.6%, respectively)
an increase in the duration of clinical benefit in patients receiving clinical benefit (median of 16.6 months vs 13.9 months, respectively).

There was also a trend towards improved survival in the fulvestrant 500 mg group (median of 25.1 months compared with 22.8 months in the 250 mg group), indicating that the observed treatment comparison for overall survival supports the advantage observed for TTP and suggesting that the benefit provided by treatment, in terms of progression, is maintained past progression.

In the subgroup of patients where it was measured, on-treatment HRQoL remained stable while patients were receiving study treatment; there was no detrimental effect of the fulvestrant 500 mg dose compared with 250 mg.

In the registration trials for fulvestrant, Studies 20/21, fulvestrant 250 mg was shown to be non-inferior to anastrozole (Robertson et al 2003). Demographic characteristics of patients in the CONFIRM study were broadly similar to those of patients in the combined analysis of Studies 20/21 and the efficacy results for fulvestrant 250 mg were consistent across the studies (median TTP of 5.5 months in CONFIRM and the combined analysis of Studies 20/21). Data from these studies give further reassurance of the significant benefit that fulvestrant 500 mg offers over an already effective 250 mg dose.

The treatment effect for TTP, favouring fulvestrant 500 mg, was consistent across all subgroups analysed. The consistency of the TTP treatment effect in the aromatase inhibitor (AI) and antioestrogen (AO) subgroups is of particular interest, given that in many markets the current regulatory approval for fulvestrant 250 mg is limited to patients who have progressed on AO therapy. Since the first regulatory approval for the use of non-steroidal AIs in breast cancer, changes in clinical practice have meant that there has been a considerable increase in the proportion of patients being treated upfront with these drugs in both the adjuvant and the advanced setting (see National Comprehensive Cancer Network [NCCN], Inc. 2009 and references therein for more details). There are few endocrine treatment options available to patients who progress on AI therapy and it is therefore important to identify agents that effectively prolong the time to progression after failing on such therapy. Although guidelines like NCCN support the use of a same class agent with a steroidal structure (steroidal AIs) in patients who have progressed on a non-steroidal AI, there are currently no agents of this type with regulatory approval for this treatment sequence. Fulvestrant 500 mg has a different mechanism of action to AIs and is the first agent to show consistent benefit in a phase III setting in patients who have progressed during either AO or AI therapy.

The safety profile of fulvestrant 500 mg is consistent with the known safety profile of fulvestrant 250 mg with no evidence for dose dependence for any AE. The 2 SAEs that were considered by the investigator to be possibly causally related to study treatment were confounded by other factors in the patients' medical histories and concomitant medications. The incidence of pre-specified AEs was well balanced between the 2 treatment groups. Although the incidence of injection site reactions was similar between treatment groups, a full assessment of the injection procedure was not possible to evaluate due to the double blind design. However, it is reassuring to observe that there is no increase in the AE incidence with doubling the dose of fulvestrant.

Overall, fulvestrant 500 mg provides improved efficacy without any detrimental effect on safety, tolerability or HRQoL compared with fulvestrant 250 mg.

Overall Conclusions

The CONFIRM study demonstrated a clear improvement in the efficacy of fulvestrant 500 mg when compared with the currently approved dose of fulvestrant 250 mg. There was a statistically significant prolongation of the TTP with a 20% reduction in the risk of progressing for patients receiving fulvestrant 500 mg. Given the superior efficacy, similar safety, tolerability and HRQoL that fulvestrant 500 mg offers over fulvestrant 250 mg we conclude that there is a superior benefit-risk profile for fulvestrant 500 mg in patients recurring or progressing on endocrine therapy.

References

Addo et al 2002

Addo S, Yates R A, Laight A. A phase I trial to assess the pharmacology of the new oestrogen receptor antagonist fulvestrant on the endometrium in healthy postmenopausal volunteers. Br J Cancer 2002;87:1354-9.

Beatson 1896

Beatson G T. On the treatment of inoperable cases of carcinoma of the mamma; suggestions for a new method of treatment with illustrative cases. Lancet 1896; 2:104-7.

Chia et al 2008

Chia S, Gradishar W, Mauriac L, Bines J, Amant F, Federico M, et al. A double blind, randomized placebo controlled trial of fulvestrant versus exemestane following prior non-steroidal aromatase inhibitor therapy in post-menopausal women with hormone receptor positive advanced breast cancer: results from EFECT. J Clin Oncol 2008; 26: 1664-70.

Collet 1997

Collet D. Modelling Survival Data in Medical Research. Chapman and Hall, 1997.

Dancey et al 2009

Dancey J E, Dodd L E, Ford R, Kaplan R, Mooney M, Rubinstein L et al.

Recommendations for the assessment of progression in randomised cancer treatment trials. European Journal of Cancer 2009;45:281-289.

DeFriend et al 1994

DeFriend D J, Howell A, Nicholson R I, Anderson E, Dowsett M, Mansel R E, et al. Investigation of a new pure antiestrogen (ICI 182780) in women with primary breast cancer. Cancer Res. 1994;54:408-414.

Dodd et al 2008

Dodd L E, Korn E L, Freidlin B, Jaffe C C, Rubinstein L V, Dancey J, et al. Blinded independent central review of progression-free survival in phase III clinical trials: important design element or unnecessary expense? J Clin Oncol. 2008;26:3791-6.

Ellis et al 2008

Ellis S, Carroll K J, Pemberton K. Analysis of duration of response in oncology trials, Contemp Clin Trials 2008; 29:456-65.

Fisher et al 2001

Fisher B, Anderson S, Tan-Chiu E, Wolmark N, Wicherham D L, Fisher E R, et al. Tamoxifen and chemotherapy for axillary node-negative, estrogen receptor-negative breast cancer findings from the National Surgical Adjuvant Breast and Bowel Project B-23. J Clin Oncol. 2001;19(4):931-42.

Howell et al 2000

Howell A, Osborne C K, Morris C, Wakeling A E. ICI 182,780 (FASLODEX): development of a novel, “pure” anti-estrogen. Cancer 2000;89:817-25.

Howell et al 2002

Howell A, Robertson J F R, Quaresma Albano J, et al. Fulvestrant, formerly ICI 182,780, is as effective as anastrozole in postmenopausal women with advanced breast cancer progressing after prior endocrine treatment J Clin Oncol, 2002; 20: 3396-3403.

Howell et al 2005

Howell A, Pippen J, Elledge R M, Mauriac L, Vergote I, Jones S E, et al. Fulvestrant versus anastrozole for the treatment of advanced breast carcinoma: a prospectively planned combined survival analysis of two multicenter trials. Cancer 2005; 104: 236-239.

Mcpherson et al 2000

McPherson K, Steel C M, Dixon J M. ABC of breast diseases. Breast cancer—epidemiology, risk factors and genetics. BMJ 2000;321:624-8.

National Comprehensive Cancer Network [NCCN], Inc. 2009

The NCCN Clinical Practice Guidelines in Oncology™ Breast Cancer (Version 1.2009)® Available at: NCCN.org. Accessed [Jun. 22, 2009]. To view the most recent and complete version of the NCCN Guidelines, go online to NCCN.org.

Osborne et al 2002

Osborne C K, Pippen J, Jones S E, et al. Double-blind, randomized trial comparing the efficacy and tolerability of fulvestrant versus anastrozole in postmenopausal women with advanced breast cancer progressing on prior endocrine therapy: Results of a North American trial. J Clin Oncol, 2002; 20: 3386-3395.

Robertson et al 2001

Robertson J F, Nicholson R I, Bundred N J, Anderson E, Rayter Z, Dowsett M, et al,. Comparison of the store-term biological effects of 7alpha-[9-(4,4,5,5,5-pentafluoropentylsulfinyl)-nonyl]estra-1,3,5, (10)-triene-3, 17beta-diol (FASLODEX) versus tamoxifen in postmenopausal women with primary breast cancer. Cancer Res 2001;6:6739-46.

Robertson et al 2003

Robertson J F R, Osborne C K, Howell A, Jones S E, Mauriac L, Ellis M, Kleeberg U R, Come S E, Vergote I, Gertler S, Buzdar A, Webster A, Morris C. Fulvestrant versus anastrozole for the treatment of advanced breast carcinoma in postmenopausal women. A prospective combined analysis of two multicenter trials. Cancer 2003;98(2):229-38.

Wakeling et al 1991

Wakeling A E, Dukes M, Bowler J. A potent specific pure anti-estrogen with clinical potential. Cancer Research 1991;51:3867-73.

Whitehead 1989

Whitehead J. The analysis of relapse clinical trials, with application to a comparison of two ulcer treatments. Stat Med 1989; 8(12):1439-54.

Wiebe et al 1993

Weibe V J, Osborne C K, Fuqua S A W, D E, Gregoria M W. Tamoxifen resistance in breast cancer. Crit Rev Oncol Hematol 1993:14:173-88.

1. A method for treating advanced breast cancer, comprising administering a composition comprising fulvestrant at a dosage of about 500 mg to a postmenopausal woman whose cancer has progressed or recurred on endocrine therapy.

2. The method according to claim 1, wherein the fulvestrant is administered monthly.

3. The method according to claim 2, wherein an additional dose of 500 mg is administered during the first month of treatment.

4. The method according to claim 3₁ wherein the additional dose is administered at about day 14.

5. The method according to claim 1, wherein the woman is oestrogen receptor positive or progesterone receptor positive.

6. The method according claim 5, wherein the woman is oestrogen receptor positive.

7. The method according to claim 1, wherein the progression or recurrence on endocrine therapy comprised therapy with tamoxifen or an aromatase inhibitor.

8. The method according to claim 7, wherein the aromatase inhibitor is selected from anastrozole, letrozole, and exemestane.

9. The method according to claim 1, wherein the administration of fulvestrant at a dosage of 500 mg increases the time to progression compared with the administration of fulvestrant at a dosage of 250 mg.
